(19) United States
(12) Patent Application Publication
(10) Pub. No.: US 2024/0202274 A1
(43) Pub. Date: Jun. 20, 2024

(54) INFORMATION PROCESSING APPARATUS, IMPROVEMENT TRAJECTORY STRUCTURING METHOD AND PROGRAM

(71) Applicant: NIPPON TELEGRAPH AND TELEPHONE CORPORATION, Tokyo (JP)

(72) Inventors: Yasunori AKAGI, Tokyo (JP); Takeshi KURASHIMA, Tokyo (JP); Hiroyuki TODA, Tokyo (JP)

(21) Appl. No.: 18/555,943

(22) PCT Filed: May 26, 2021
(86) PCT No.: PCT/JP2021/020090

§ 371 Date: Oct. 18, 2023

(51) Int. Cl. G06F 17/11

(52) U.S. Cl. CPC G06F 17/11

(57) ABSTRACT

An information processing apparatus includes an input unit configured to acquire input information including an initial first numerical index and a second numerical index that serves as a goal, and an improvement trajectory structuring unit configured to structure an improvement trajectory indicating a trajectory from the first numerical index to the second numerical index in time series, based on the input information.

### TECHNICAL FIELD

The present invention relates to an information processing apparatus, an improvement trajectory structuring method, and a program.

### BACKGROUND ART

A technique for assisting a user in preparing an improved plan with numerical indices is known. In addition, for many people, it is known that the value of reward (reward such as money, joy associated with achievement of goal, and the like) obtained upon achievement of a goal decreases when the time of attainment is far away from the present. This phenomenon is called “time discounting,” for which research is conducted in fields such as behavioral economics (Non-Patent Literature 1).

### CITATION LIST

### Non-Patent Literature

Non-Patent Literature 1: J. Benhabib, B. Alberto, and S. Andrew. Present-bias, Quasi-hyperbolic Discounting, and Fixed Costs, Games and Economic Behavior 69.2 (2010): 205-223.

### SUMMARY OF INVENTION

### Technical Problem

When an improvement plan enables earlier achievement of a goal, the improvement plan has a higher current value, and there is an effect of increasing the user's motivation. However, if the indices are raised too rapidly, the burden on the user becomes quite large, and on the contrary, the motivation of the user decreases. Therefore, there is a demand for preparing an appropriate improvement plan until the goal is achieved by taking the trade-off of these into account.

An object of the technique disclosed herein is therefore to assist preparation of an appropriate improvement plan with numerical indices.

### Solution to Problem

The disclosed technique provides an information processing apparatus including an input unit configured to acquire input information including an initial first numerical index and a second numerical index that serves as a goal, and an improvement trajectory structuring unit configured to structure an improvement trajectory indicating a trajectory from the first numerical index to the second numerical index in time series, based on the input information.

### Advantageous Effects of Invention

It is possible to assist preparation of an appropriate improvement plan with numerical indices.

### BRIEF DESCRIPTION OF DRAWINGS

FIG. 1 is a functional configuration diagram of an information processing apparatus.

FIG. 2 is a diagram illustrating an example of input information.

FIG. 3 is a flowchart illustrating an example of a flow of an improvement trajectory output process.

FIG. 4 is a diagram illustrating an example of an algorithm of improvement trajectory structuring.

FIG. 5 is a diagram illustrating an example of an improvement trajectory.

FIG. 6 is a diagram illustrating a hardware configuration example of a computer.

### DESCRIPTION OF EMBODIMENTS

Hereinafter, an embodiment (present embodiment) of the present invention will be described with reference to the drawings. The embodiment to be described below is merely an example, and an embodiment to which the present invention is applied is not limited to the following embodiment.

### Outline of Present Embodiment

An information processing apparatus according to the present embodiment is a device that outputs an improvement trajectory of a numerical index based on input information including an initial numerical index (first target value) and a numerical index that serves as a goal (second target value) in a situation aiming at improving a numerical index, such as weight loss by diet or a rating system in a chess-game or programming contest.

The improvement trajectory is information indicating the trajectory, in time series, from the initial numerical index to a numerical index that serves as a goal.

Next, formulation of a problem handled in the present embodiment will be described. Input of an information processing apparatus 10 is as follows. Note that the numerical indices and times are discretized as appropriate.
(1) Initial numerical index y_{c}.
(2) Numerical index y_{g} to serve as a goal. In an example of diet, y_{g}<y_{c} is assumed, or in an example of rating, y_{g}>y_{c} is assumed. Hereinafter, description will be made under y_{g}>y_{c}, but similar processing can be performed in a case of y_{g}<y_{c}.
(3) Reward R upon achievement of a goal.
(4) Time discounting function f(t) representing the discount rate of reward unit time t later. The reward R time t later makes the user feel the value of R·f(t). Normally, a monotonically decreasing function is assumed for f(t), but a similar assumption is made in the present embodiment.
(5) Function g(y₁, y₂) representing the labor (distance) required to change the index from y₁ to y₂ per unit time. It is assumed that g(y₁, y₂)>0 at any y₁, y₂.
(6) Parameter λ for adjusting the trade-off between reward and labor. When λ is large, emphasis is placed on increasing the reward, and when λ is small, emphasis is placed on reducing the labor.

It is assumed that these values are determined in advance by the user.

Furthermore, output of the information processing apparatus 10 is an improvement trajectory that minimizes the weighted sum of the negative current value of time-discounted reward and the total distance. Specifically, the following Expression (1) is minimized by a row y₀(y_{c}), y₁, . . . , and y_{T} (=y_{g}) of the index values at time 0, 1, . . . , and T.

$$[ Math . 1 ]$$

$${\sum }_{t = 0}^{T} g ( {y}_{t} , {y}_{t + 1} ) - λ \cdot R \cdot f ( T ) \qquad ( 1 )$$

Note that the value of T is also a part of the output, and is a value determined by the information processing apparatus 10.

### (Functional Configuration of Information Processing Apparatus)

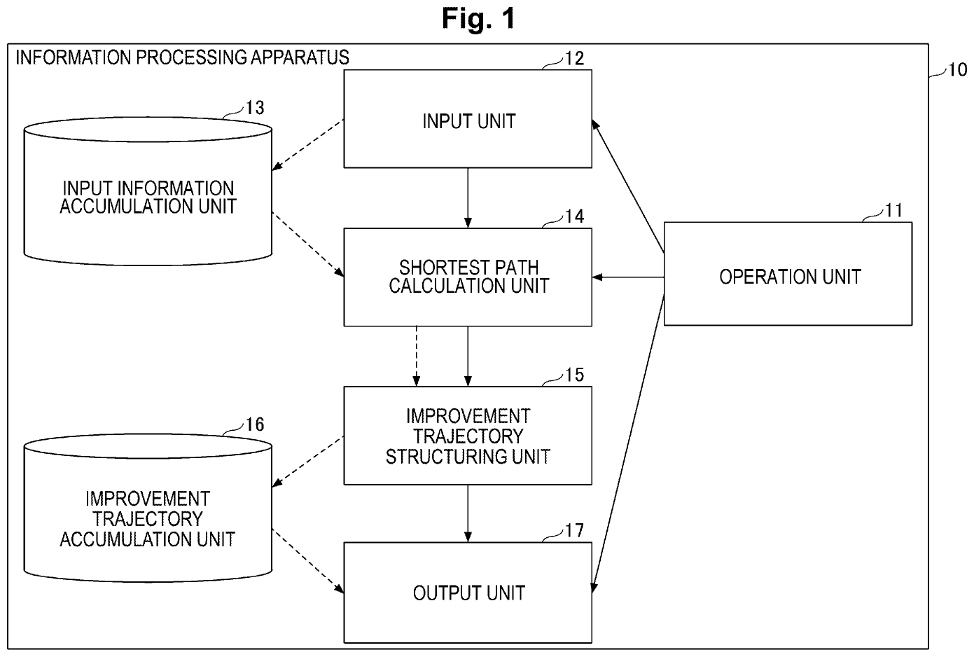

FIG. 1 is a functional configuration diagram of an information processing apparatus. The information processing apparatus 10 includes an operation unit 11, an input unit 12, an input information accumulation unit 13, a shortest path calculation unit 14, an improvement trajectory structuring unit 15, an improvement trajectory accumulation unit 16, and an output unit 17.

The operation unit 11 provides an interface for performing operations from the outside. The operation unit 11 enables operations such as storage and correction of input data by operating the input unit 12, start of calculation by sending a command to the shortest path calculation unit 14, and output of an estimation result by sending a command to the output unit 17.

The input unit 12 stores data in the input information accumulation unit 13 and corrects the data. Specific inputs include an initial numerical index, a target numerical index, a reward upon achievement of the goal, a time discounting function, a function representing the distance between the numerical indices, and a parameter for adjusting the trade-off between the reward and the distance.

The shortest path calculation unit 14 calculates the shortest path by the Dijkstra method (Reference Literature [1]) in order to structure an improvement trajectory. Details of the calculation will be described later.

The improvement trajectory structuring unit 15 structures the improvement trajectory based on the calculated shortest path. Specifically, from the obtained shortest path (y₀=y_{g}, 0), (y₁, 1), . . . , and (y_{T}=y_{c}, T), an improvement trajectory of y_{T}, y_{T-1}, . . . , and y₀ is structured. The structured improvement trajectory minimizes Expression (1).

The improvement trajectory accumulation unit 16 accumulates the structured improvement trajectories.

The output unit 17 reads and outputs the improvement trajectories stored in the improvement trajectory accumulation unit 16. The output unit 17 may display a screen showing the improvement trajectories on a display or the like, or may transmit information indicating the improvement trajectories to another device.

### (Information Handled by Information Processing Apparatus)

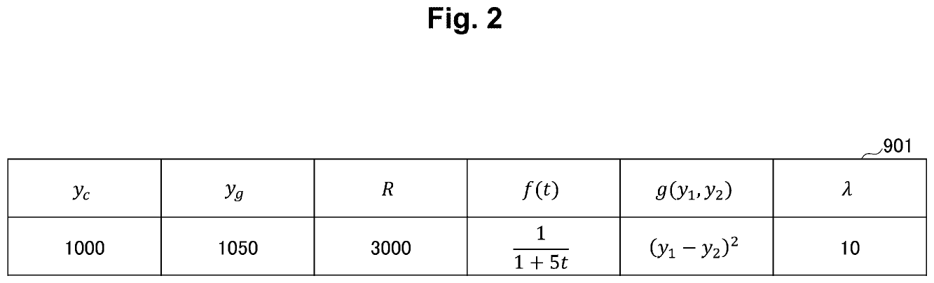

FIG. 2 is a diagram illustrating an example of input information. Input information 901 includes, as items, an initial numerical index y_{c}, a numerical index y_{g} to serve as a goal, a reward R upon achievement of the goal, a time discounting function f(t) representing the discount rate of the reward a unit time t later, a function g(y₁, y₂) representing the labor (distance) required to change the index from y₁ to y₂ in the unit time, and a parameter λ for adjusting the trade-off between the reward and the labor.

### (Operation of Information Processing Apparatus)

Next, an operation of the information processing apparatus 10 will be described with reference to the drawings. In the information processing apparatus 10, the operation unit 11 receives an operation or the like of the user and starts the improvement trajectory output process.

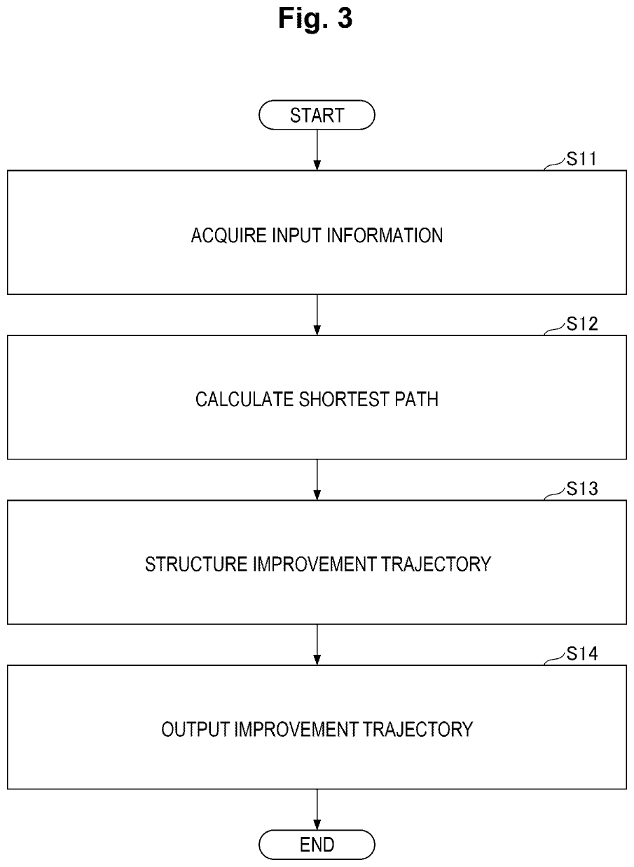

FIG. 3 is a flowchart illustrating an example of a flow of the improvement trajectory output process. The input unit 12 acquires the input information 901 (step S11). The acquired input information 901 is stored in the input information accumulation unit 13.

Next, the shortest path calculation unit 14 calculates the shortest path by applying the Dijkstra method to the input information (step S12).

Subsequently, the improvement trajectory structuring unit 15 structures an improvement trajectory based on the calculated shortest path (step S13). The structured improvement trajectory is stored in the improvement trajectory accumulation unit 16.

The output unit 17 outputs the structured improvement trajectory (step S14).

### (Details of Shortest Path Calculation)

Next, the process of the shortest path calculation unit 14 in step S12 of the improvement trajectory output process will be described in detail.

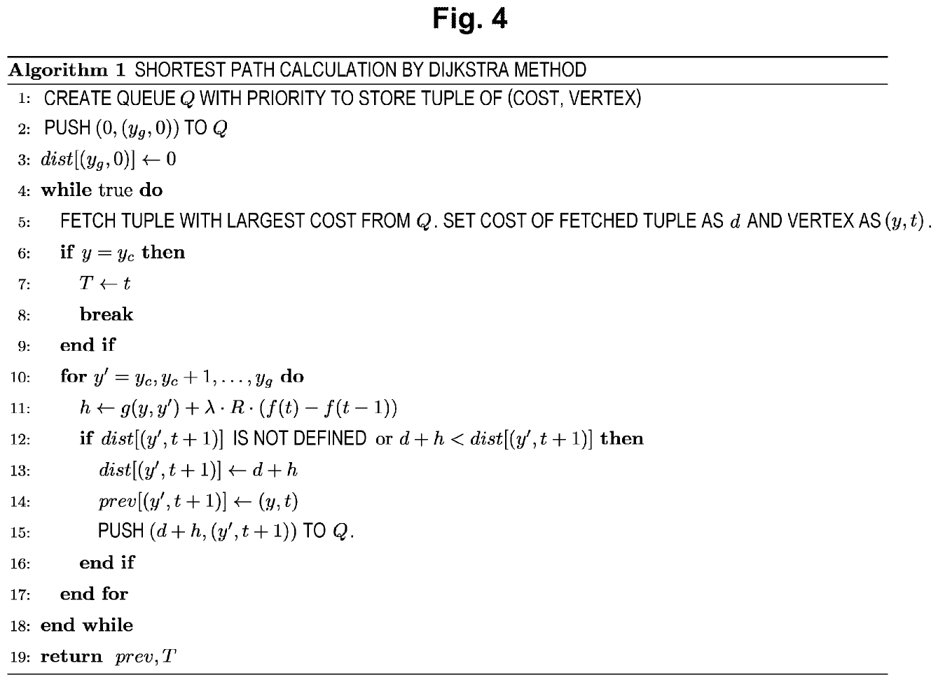

FIG. 4 is a diagram illustrating an example of an algorithm for improvement trajectory structuring. A graph in which each vertex is represented in the form of (value of index, time) will be considered. It is assumed that a side of a cost g(y, y′)+λ·R·(f(t)−f(t−1)) is drawn from a point (y, t) toward a point (y′, t+1) for each y′∈{y_{c}, y_{c}+1, . . . , y_{g}}. In this graph, the shortest path calculation unit 14 executes the Dijkstra method with the start point as (y_{g}, 0), and ends the search when the search reaches a vertex in the form of (y_{g}, T) (T is an arbitrary non-negative integer) for the first time. The shortest path calculation unit 14 calculates the shortest path by tracing back from the (y_{g}, T) using the information prev returned by this algorithm.

Since the monotonic decreasing property of f and g take only positive values, the cost of each side is always positive. This can indicate that the above algorithm outputs the shortest path from (y_{g}, 0) to (y_{g}, T), and that the above algorithm always ends.

### (Example of Structured Improvement Trajectory)

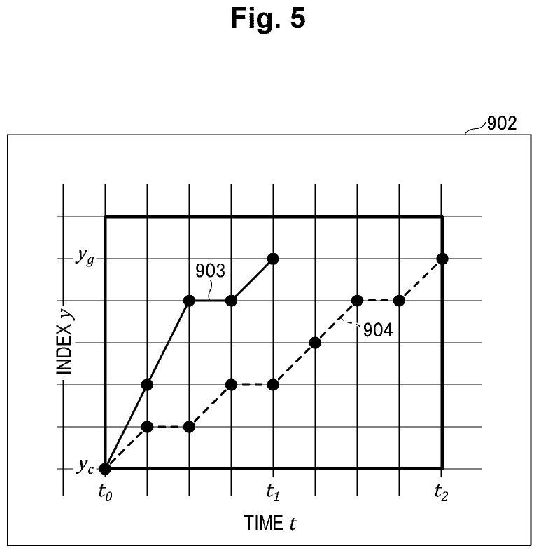

FIG. 5 is a diagram illustrating an example of an improvement trajectory. The screen 902 is an example of a screen displayed on the display by the output unit 17. For one piece of input information, one improvement trajectory is structured. The screen 902 includes the first improvement trajectory 903 and the second improvement trajectory 904, and thus displays two improvement trajectories built for the two pieces of input information respectively.

For example, the first improvement trajectory 903 and the second improvement trajectory 904 are structured by using, as inputs, indices such as the first index y_{c} at the initial time to and the second index y_{g} to serve as a goal. Since the reward R, the time discounting function f(t), the function g(y₁, y₂), and the parameter λ are different from each other, different improvement trajectories are structured.

### (Hardware Configuration Example of Computer)

Each functional unit of the information processing apparatus 10 described above can be implemented by causing a computer to execute a program describing processing contents described in the present embodiment. Note that the “computer” may be a physical machine or may be a virtual machine in a cloud. In case a virtual machine is used, “hardware” to be described herein is virtual hardware.

The above program is recorded in a computer-readable recording medium (such as a portable memory) so that the program can be stored and distributed. Also, the program can be provided through a network such as the Internet or an electronic mail.

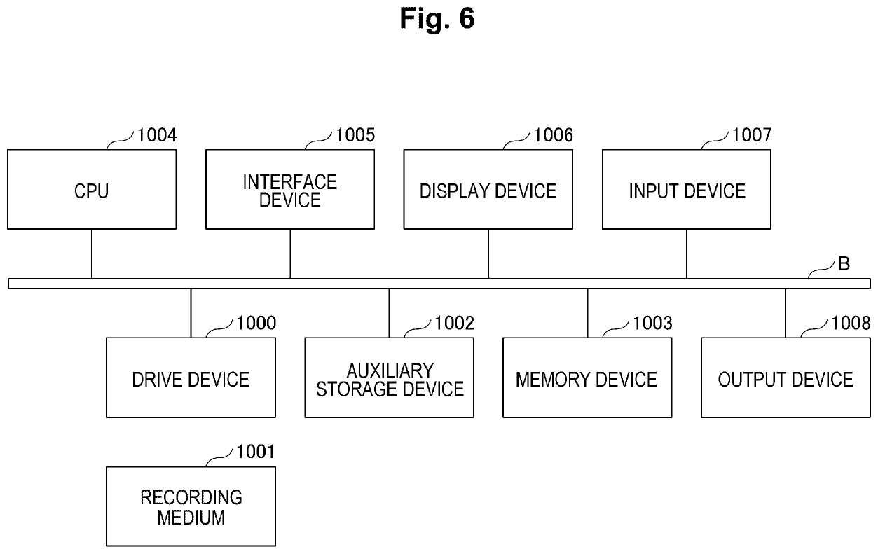

FIG. 6 is a diagram illustrating a hardware configuration example of the computer. The computer in FIG. 6 includes a drive device 1000, an auxiliary storage device 1002, a memory device 1003, a CPU 1004, an interface device 1005, a display device 1006, an input device 1007, an output device 1008, and the like, which are connected to each another by a bus B.

The program for implementing the processing in the computer is provided by, for example, a recording medium 1001 such as a CD-ROM or a memory card. When the recording medium 1001 that stores the program is set in the drive device 1000, the program is installed from the recording medium 1001 into the auxiliary storage device 1002 via the drive device 1000. However, the program is not necessarily installed from the recording medium 1001, and may be downloaded from another computer via a network. The auxiliary storage device 1002 stores the installed program, and also stores necessary files, data, and the like.

When a command to start the program is issued, the memory device 1003 reads the program from the auxiliary storage device 1002, and stores the program therein. The CPU 1004 implements the functions related to the information processing apparatus, according to the program stored in the memory device 1003. The interface device 1005 is used as an interface for connecting with a network. The display device 1006 displays a graphical user interface (GUI) or the like according to the program. The input device 1007 includes a keyboard and a mouse, buttons, a touch panel, and the like, and is used to input various operation commands. The output device 1008 outputs a calculation result. Note that the computer may include a graphics processing unit (GPU) or a tensor processing unit (TPU) instead of the CPU 1004 and may include the GPU or TPU in addition to the CPU 1004. In that case, for example, the processing may be shared and executed such that the GPU or the TPU executes processing requiring special computation and the CPU 1004 executes other processing.

### Effects of Present Embodiment

With the information processing apparatus 10 according to the present embodiment, the shortest path calculation by the Dijkstra method is performed based on input information, and the improvement trajectory that minimizes the weighted sum of the initial value of the time-discounted negative reward and the total distance is structured based on the calculated shortest path. Thus, it is possible to assist preparation of an appropriate improvement plan with numerical indices in which the trade-off between the reward and the distance is adjusted.

For example, when the user recognizes an improvement trajectory output from the output unit 17, it is possible to assist the improvement plan by the user. In addition, by outputting the improvement trajectory to a goal management system or the like, the goal managed by the goal management system or the like can be detailed, and the goal including the progress can be managed.

### REFERENCE LITERATURE

[1] R. K. Ahuja, T. L. Magnanti, J. B. Orlin, Network Flows: Theory, Algorithms, Applications, Prentice Hall, 1993.

### Summary of Embodiment

In the present specification, an information processing apparatus, an improvement trajectory structuring method, and a program described in at least the following items are described.

### (Item 1)

An information processing apparatus, including:
an input unit configured to acquire input information including an initial first numerical index and a second numerical index that serves as a goal; and
an improvement trajectory structuring unit configured to structure an improvement trajectory indicating a trajectory from the first numerical index to the second numerical index in time series, based on the input information.

### (Item 2)

The information processing apparatus according to item 1,
in which the input information further includes a reward for achieving the goal, a time discounting function, a function representing labor required to change a numerical index in a unit time, and a parameter for adjusting a trade-off between the reward and the labor, and
in which the improvement trajectory structuring unit structures the improvement trajectory that minimizes a weighted sum of a negative current value of a time-discounted reward and a total distance.

### (Item 3)

The information processing apparatus according to item 1 or 2, further including a shortest path calculation unit configured to calculate a shortest path by Dijkstra method based on the input information,
in which the improvement trajectory structuring unit structures the improvement trajectory based on the calculated shortest path.

### (Item 4)

An improvement trajectory structuring method executable on a computer, the method including:
acquiring input information including an initial first numerical index and a second numerical index that serves as a goal; and
structuring an improvement trajectory indicating a trajectory from the first numerical index to the second numerical index in time series, based on the input information.

### (Item 5)

A program for causing a computer to function as each unit in the information processing apparatus according to any one of items 1 to 3.

Although the present embodiment has been described above, the present invention is not limited to such a particular embodiment, and various modifications and changes can be made within the scope of the gist of the present invention described in the claims.

### REFERENCE SIGNS LIST

10 Information processing apparatus
11 Operation unit
12 Input unit
13 Input information accumulation unit
14 Shortest path calculation unit
15 Improvement trajectory structuring unit
16 Improvement trajectory accumulation unit
17 Output unit
1000 Drive device
1001 Recording medium
1002 Auxiliary storage device
1003 Memory device
1004 CPU
1005 Interface device
1006 Display device
1007 Input device
1008 Output device

1. An information processing apparatus, comprising:
an input unit configured to acquire input information including an initial first numerical index and a second numerical index that serves as a goal; and
an improvement trajectory structuring unit configured to structure an improvement trajectory indicating a trajectory from the first numerical index to the second numerical index in time series, based on the input information.

2. The information processing apparatus according to claim 1,
wherein the input information further includes a reward for achieving the goal, a time discounting function, a function representing labor required to change a numerical index in a unit time, and a parameter for adjusting a trade-off between the reward and the labor, and
wherein the improvement trajectory structuring unit structures the improvement trajectory that minimizes a weighted sum of a negative current value of a time-discounted reward and a total distance.

3. The information processing apparatus according to claim 1, further comprising a shortest path calculation unit configured to calculate a shortest path by Dijkstra method based on the input information,
wherein the improvement trajectory structuring unit structures the improvement trajectory based on the calculated shortest path.

4. An improvement trajectory structuring method executable on a computer, the method comprising:
acquiring input information including an initial first numerical index and a second numerical index that serves as a goal; and
structuring an improvement trajectory indicating a trajectory from the first numerical index to the second numerical index in time series, based on the input information.

5. A non-transitory computer-readable recording medium storing a program that, when executed on a computer, causes the computer to function as each unit in the information processing apparatus according to claim 1.
